(19) United States
(12) Patent Application Publication
(10) Pub. No.: US 2021/0172950 A1
(43) Pub. Date: Jun. 10, 2021

(54) ISOLATING AND ANALYZING RARE BRAIN-DERIVED CELLS AND PARTICLES

(71) Applicant: The General Hospital Corporation, Boston, MA (US)

(72) Inventors: Mehmet Toner, Charlestown, MA (US); Cenk Ayata, Sudbury, MA (US); Tao Qin, North Reading, MA (US); Nezihi Murat Karabacak, Arlington, MA (US)

(21) Appl. No.: 17/255,258

(22) PCT Filed: Jun. 28, 2019
(86) PCT No.: PCT/US2019/039816

§ 371 Date: Dec. 22, 2020

Related U.S. Application Data
(60) Provisional application No. 62/692,289, filed on Jun. 29, 2018.

(51) Int. Cl. G01N 33/569; G01N 33/543; G01N 33/68

(52) U.S. Cl. CPC G01N 33/56972; G01N 33/54326; G01N 2800/2871; G01N 33/6896; G01N 33/54366

(57) ABSTRACT

This disclosure relates to systems and methods for isolating, detecting, and/or analyzing brain-derived cells or particles in the blood circulation of human and animal subjects.

### CROSS-REFERENCE TO RELATED APPLICATIONS

This application is an international application of and claims priority to U.S. Provisional Application Ser. No. 62/692,289, filed on Jun. 29, 2018, the entire contents of which are hereby incorporated by reference.

### TECHNICAL FIELD

This disclosure relates to isolating and analyzing rare brain-derived cells and particles, and in particular, brain-derived cells and particles circulating in the bloodstream.

### BACKGROUND

Concussion and mild traumatic brain injury (TBI) have tremendous clinical and socioeconomic impact in sports and the military. Our pathophysiological understanding, diagnosis, and management of concussion have long suffered from a lack of diagnostic tools. Neuroimaging lacks sensitivity and specificity, requires hospital settings, and is expensive and time consuming. Other neurodiagnostic modalities (EEG, lumbar puncture, etc.) are of limited utility. Small molecules or peptides released from the brain into the blood have long been sought as biomarkers, but none has achieved sufficient sensitivity and specificity to enter clinical practice until recently. Today, the diagnosis and management of concussion relies purely on clinical judgment, which is both insensitive and non-specific. Therefore, accessible biomarkers of TBI have been an unmet need, a “holy grail,” for decades. Similarly, the proper diagnosis of other brain injuries due to disease has also had limited success.

### SUMMARY

The present disclosure provides new point-of-care technology to diagnose concussion and mild traumatic brain injury (TBI) at the bedside based on circulating brain-derived cells, cell clusters, and particles, such as brain-derived endothelial cells (BECs), neurons, microglia, astrocytes, extracellular vesicles, exosomes, and organelles. The technology will enable observations not previously possible (i.e., the observation of previously invisible BECs), reveal novel biomarkers, and open new avenues with broad applications even outside the TBI field, potentially matching its impact in cancer.

In one aspect, the disclosure features methods for isolating and/or analyzing brain-derived cells or particles, such as brain-derived cells such as endothelial cells (BECs), neurons, microglia, and astrocytes, and brain-derived particles such as organelles or extracellular vesicles, e.g., microvesicles (MVs), exosomes, oncosomes, and apoptotic bodies, from a blood sample from a subject.

The methods include obtaining a blood sample from the subject; mixing the blood sample with magnetic beads including a binding agent that specifically binds to white blood cells (WBCs) and not to the other cells or particles, for a time and under conditions sufficient for the binding agent to bind to the WBCs; flowing the blood sample through a first module comprising a microfluidic size-based separation system configured to direct small cells and particles such as red blood cells (RBCs) and platelets in the blood sample to a first waste outlet and to direct the remaining blood sample to a second module comprising an inertial focusing channel; flowing the remaining blood sample through the second module at a flow rate and for a distance sufficient to cause cells and/or particles in the remaining blood sample to align in one or more streamlines within the remaining blood sample flowing in the inertial focusing channel; flowing the remaining blood sample with the cells and/or particles aligned in one or more streamlines through a third module comprising a magnetophoresis system for a time and distance sufficient to separate WBCs bound to magnetic beads from cells and particles not bound to magnetic beads, and flowing the WBCs into a second waste outlet and flowing other cells and particles to a product outlet; obtaining cells or particles from the product outlet and determining which of the cells or particles originate in brain tissue; and analyzing the brain-derived cells or particles.

Analyzing cells, such as BECs, as described herein, e.g., using droplet digital polymerase chain reaction (ddPCR) can also be done with cells that are isolated using other known methods of isolation.

In another aspect, the disclosure provides methods of isolating and/or analyzing brain-derived cells or particles, such as BECs, neurons, microglia, and astrocytes, and brain-derived particles such as organelles or extracellular vesicles, e.g., microvesicles (MVs), exosomes, oncosomes, and apoptotic bodies, from a blood sample from a subject. These methods include obtaining a blood sample from the subject; mixing the blood sample with magnetic beads comprising a binding agent that specifically binds to one or more specific types of cells or specific types of particles and not to white blood cells (WBCs), for a time and under conditions sufficient for the binding agent to bind to the brain-derived cells or particles; flowing the blood sample through a first module comprising a microfluidic size-based separation system configured to direct small cells and particles such as red blood cells (RBCs) and platelets in the blood sample to a first waste outlet and to direct the remaining blood sample to a second module comprising an inertial focusing channel; flowing the remaining blood sample through the second module at a flow rate and for a distance sufficient to cause cells and/or particles in the remaining blood sample to align in one or more streamlines within the remaining blood sample flowing in the inertial focusing channel; flowing the remaining blood sample with the cells and/or particles aligned in one or more streamlines through a third module comprising a magnetophoresis system for a time and distance sufficient to separate the specific types of cells or particles bound to magnetic beads from WBCs cells, other cells, and particles not bound to magnetic beads, and flowing the WBCs other cells, and particles not bound to magnetic beads into a second waste outlet and flowing the bound cells or particles to a product outlet; obtaining bound cells or particles from the product outlet and determining which of the bound cells or particles originate in brain tissue; and analyzing the brain-derived cells or particles.

In some of these methods, the brain-derived cells include one or more of brain-derived endothelial cells (BECs), neurons, microglia, and astrocytes. In other of these methods, the brain-derived particles comprise extracellular vesicles, e.g., one or more of microvesicles (MVs), exosomes, oncosomes, and apoptotic bodies.

In certain implementations, the first module includes an inertial exchanger configured to direct small cells such as red blood cells and platelets and particles in the blood sample to a first waste outlet and to direct the remaining blood sample to the second module. Alternatively, the first module can be or include a deterministic lateral displacement array of microposts in a channel, wherein the array of microposts is configured to direct small cells such as red blood cells and platelets and particles in the blood sample to a first waste outlet and to direct the remaining blood sample to the second module.

In any of these methods, determining whether a cell or particle originated in brain tissue includes analyzing the cell or particle using droplet digital PCR, an immunoassay, or both. Alternatively, determining whether a cell or particle originated in brain tissue includes analyzing the cell or particle using detection of antigens unique to brain-derived cells or particles via fluorescently conjugated antibodies. In some embodiments, determining whether a cell or particle originated in brain tissue includes analyzing the cell or particle using brain-specific genes, transcripts, or proteins for differentiating brain-derived cells or particles from cells or particles of non-cerebral origin. In other embodiments, determining whether a cell or particle originated in brain tissue includes analyzing the cell or particle using single-cell RNA sequencing. In any of these methods, the brain-derived cells can be one or more of brain-derived endothelial cells (BECs), neurons, microglia, and astrocytes. In some embodiments, the brain-derived particles can be or include extracellular vesicles.

In some embodiments, the brain-specific genes include occludin and promininl, and wherein transcripts of these genes are used to detect brain-derived cells comprising brain-derived endothelial cells (BECs).

In various implementations of these methods, the subject, such as a human or animal (e.g., cat, dog, mouse, rat, rabbit, monkey, ape, pig, cow sheep, goat, or horse) subject, has a brain disorder selected from the group consisting of mild, moderate, or severe traumatic brain injury, vascular brain injury (selected from the group consisting of primary CNS vasculitis, acute focal cerebral ischemia, and small vessel disease), and neurodegenerative disease (selected from the group consisting of Alzheimer's disease, Parkinson's disease, and amyotrophic lateral sclerosis).

In some embodiments, the methods can further include detecting a quantity of the brain-derived cells or particles, e.g., BECs, a quality of the brain-derived cells or particles, or both, for detecting a specific type of brain disorder or damage to the blood brain barrier.

In some implementations, the magnetic beads specifically bind to WBCs and not to endothelial cells or the magnetic beads specifically bind to endothelial cells and not to WBCs. Any of these methods can further include separating the brain-derived cells, e.g., BECs, or brain-derived particles, from other cells and/or particles in the blood sample to isolate the brain-derived cells or particles.

In another aspect, the disclosure features systems for analyzing and/or isolating brain-derived cells or particles, such as brain-derived cells such as endothelial cells (BECs), neurons, microglia, and astrocytes, and brain-derived particles such as organelles or extracellular vesicles, e.g., microvesicles (MVs), exosomes, oncosomes, and apoptotic bodies, from a blood sample from a subject.

These systems as described herein include a mixer for combining the blood sample with magnetic beads including a binding agent that specifically binds to either (i) brain-derived cells or particles and not to white blood cells (WBCs), or (ii) WBCs and not to other cells or particles, for a time and under conditions sufficient for the binding agent to bind; a first module including a microfluidic size-based separation system configured to direct small cells and particles such as red blood cells and platelets in the blood sample to a first waste outlet; a second module including an inertial focusing channel, wherein the remaining blood sample is controlled to flow through the second module at a flow rate and for a distance sufficient to cause cells and particles in the remaining blood sample to align in one or more streamlines within the remaining blood sample flowing in the inertial focusing channel; a third module including a magnetophoresis system configured to separate cells or particles bound to magnetic beads from cells and particles not bound to magnetic beads, thus separating bound cells or particles from unbound cells and/or particles and flowing the WBCs into a second waste outlet and flowing unbound cells and/or particles to a product outlet; and a fourth module including a cell or particle analyzer configured to determine which of the cells or particles originate in brain tissue and, optionally, to separate cells or particles originating in brain tissue from cells or particles originating in other tissues, thereby analyzing and/or isolating brain-derived cells or particles from the blood sample.

In some implementations of these systems, the first module is or includes an inertial exchanger configured to direct small cells and particles such as red blood cells and platelets in the blood sample to a first waste outlet and to direct the remaining blood sample to the second module. In other implementations, the first module is or includes a deterministic lateral displacement array of microposts in a channel, wherein the array of microposts is configured to direct small cells and particle such as red blood cells and platelets in the blood sample to a first waste outlet and to direct the remaining blood sample to the second module.

In some implementations, the fourth module is or includes a system to encapsulate cells or particles in individual droplets and to perform ddPCR on each individual droplet to determine which cells or particles originate from brain tissue.

Previous studies on cBECs have relied on flow cytometry with fluorescence-activated cell sorting (FACS), with poor sensitivity, high cost, and lack of bedside potential. The present disclosure describes systems and methods that overcome these barriers and capture ultra-rare cBECs by employing microfluidic technology with unprecedented sensitivity, cost-effectiveness, and bedside point-of-care potential. The new systems and methods can be used to capture ultra-rare cBECs with high sensitivity in limited volume blood samples, differentiate cBECs from other circulating endothelial cells of non-cerebral origin (cEC), and can be used to elucidate the relationship between mild TBI and cBECs as well as other brain disorders including traumatic (moderate and severe traumatic brain injury), vascular (primary central nervous system (CNS) vasculitis, acute focal cerebral ischemia, and small vessel disease) and neurodegenerative diseases (Alzheimer's disease, Parkinson's disease, and amyotrophic lateral sclerosis).

Once the nature of a relationship is established as diagnostic for the specific brain injury or disorder, different clinical options are provide that depend on patient condition. For concussions, accurate and timely diagnosis is critical in the management of patients, since it ensures swift treatment prevention of recurrences (especially in sports-related concussions) as repeated concussions within a critical time window lead to significantly worse outcomes. In brain diseases, where treatment options are available (e.g., lessening of cognitive symptoms in Alzheimer's disease), continuous monitoring of treatment side effects with the invention will be valuable for managing the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic representation of an example of a microfluidic chip as described herein.

FIG. 2 is a schematic representation of an example of a microfluidic chip as described herein and illustrating the key steps to capture brain endothelial cells (BECs).

FIG. 3A is a schematic diagram of a so-called “iChip” as described herein that initially separates the flow equally into two parallel and equivalent channels for higher throughput. Cells are tightly focused in the inertial focusing regions and then separated in the magnetophoresis (deflection) regions. The first magnetophoresis stage is designed to have a relatively lower magnetic gradient for bulk depletion: only WBC with >7 magnetic beads are deflected.

FIG. 3B is a series of representations of fluorescent cell streaks formed during iChip operation showing inertial focusing and magnetophoresis of cells with (green, bottom trace in first three images and top trace in the last image on the right) or without (orange, top trace in first three images and bottom trace in the last image on the right) magnetic load.

FIG. 4 is a schematic representation of examples of steps in RNA quantification using the iChip and droplet digital PCR (ddPCR).

FIG. 5 is a representation of an experimental design to test whether an iChip can capture BECs in whole blood. Individual steps are described in the text.

FIG. 6A is a series of green fluorescent protein (GFP)+ BECs (arrowheads) in an iChip product. Non-GFP cells are also present (DAPI nuclear stain).

FIG. 6B is a graph of BEC counts in 1 ml whole blood (In) and in iChip product (Out).

FIG. 7 is a schematic representation of an experimental design to test whether an iChip as described herein can capture circulating BECs (cBECs) from whole blood.

FIG. 8 is a bar graph of whole blood (including cBEC) captured in an iChip product after intravenous injection of three different dose levels of fluorescent BECs, circulating between 5-960 minutes in recipient mice. Sample sizes (N) shown on each bar represent number of mice.

FIGS. 9A-9D is a series of graphs of multichannel flow cytometry/FACS, which demonstrate expression of four surface markers in CD31+/PI− ECs from brain, lung, and liver.

FIG. 10 is chart of the number of reads obtained using RNAseq to select transcripts for obtaining “cBEC burden.”

FIG. 11 is a graph showing RNA sequencing average number of reads for candidate markers of cBECs (left side with <1 lung EC reads) and cECs (right side). A median with an interquartile range is shown.

FIG. 12 is a schematic representation of an experimental scheme for testing sensitivity and specificity of the combined use of an iChip and ddPCR as described herein.

FIGS. 13A-13D are a series of four graphs that show ddPCR transcript counts in brain (red circles), liver (blue triangles), and lung ECs (green square), and naïve whole blood (pink diamonds) in an iChip product.

FIG. 14 is a series of linked graphs showing the results of an experiment to test combinations of iChip and ddPCR in which mouse brain or lung cell suspensions containing endothelial cells (or no cells) were spiked into mouse blood samples.

FIG. 15 is a graph showing that a single severe CHI (red dots) acutely sheds ECs into the circulation. Yellow dots represent naïve, sham, and CCI results. Each data point on the graph represents a single animal.

FIGS. 16A-16E are a series of bar graphs showing results of an experiment where mouse closed head injury (TBI model) or musculoskeletal injury (sham) were performed to test changes in the quantity of cBECs and cECs. These graphs show medians with interquartile ranges.

FIGS. 17A-17E are a series of graphs showing results of an experiment where mouse ischemia (stroke model) was performed to test changes in the quantity of cBECs and cEC (showing medians with interquartile ranges).

### DETAILED DESCRIPTION

In this disclosure, we challenge the notion that brain cells and particles stay within the brain. To the contrary, we have found that traumatic brain injury (TBI) leaves “footprints” in the blood by shedding brain endothelial cells (BEC) and other cells and particles into the systemic circulation. The brain is the most densely vascularized organ, receiving 20% of the cardiac output despite being only 2% of body weight. The entire human blood volume circulates through the brain once every 3-5 minutes and is exposed to about 14 million brain endothelial cells per gram of brain tissue. Due to their proximity to circulating blood, BECs and other cells and particles are shed into the circulation, and thus a change in the concentration, surface markers, and gene expression of circulating BECs (cBECs) provide information relating to TBI.

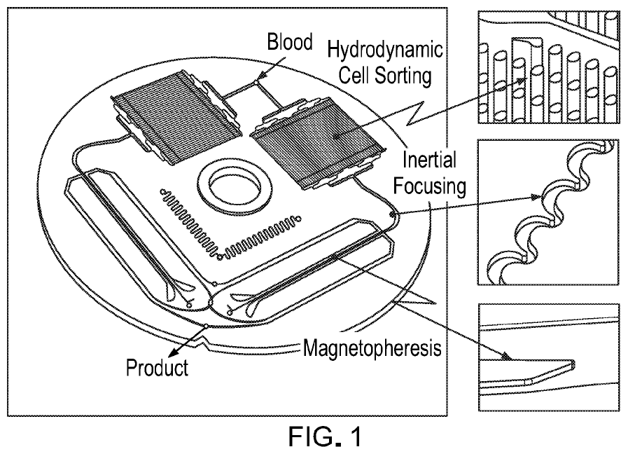

As described herein, microfluidic technology has been developed to transform the diagnosis and management of TBI. The microfluidic chip (FIG. 1), elements of which are described, for example, in U.S. Pat. Nos. 8,784,012; 9,610,582; and 9,808,803; and 9,895,694; and US Published Patent Application No. US2016/0123858; which are all incorporated herein by reference in their entireties, uses a first module that uses size-based separation, for example, negative depletion by size-based hydrodynamic cell sorting (i.e., deterministic lateral displacement), a second module having an inertial focusing channel, and a third module that includes a magnetophoresis system. The new systems can process high volumes of blood, e.g., 20 cc of blood in 30 minutes, to find extremely rare target cells, e.g., one target cell in 10⁹ blood cells (i.e., can find a needle in a haystack). Moreover, captured cells are healthy, and can be used for molecular analyses such as single cell RNA sequencing (scRNAseq).

In other embodiments, the systems described herein can also be run in a positive selection mode to isolate the target cells directly.

The new systems are portable and affordable point-of-care devices. The new systems and methods will provide a significant clinical impact. For example, detecting the circulating BEC relating to concussion on a sports field (2-3 million in US/year (Langlois, Rutland-Brown et al. 2006)) in the battlefield (˜250,000 in US since 2000 (Helmick, Spells et al. 2015)), or in emergency rooms will save billions of dollars every year in timely diagnosis and management, and help specific measures to be taken to prevent repeat concussions and numerous post-concussive health problems. Thus, the presently disclosed microfluidic technology can transform the research, diagnosis, and management of concussion by allowing large scale screening for and early detection of high-risk individuals, guiding treatment selections, and monitoring treatment efficacy.

### Brain-Derived Cells and Particles

Cerebral vasculature serves much more than plumbing for the brain; it also forms an interface and a barrier between the brain and the circulating blood. Brain-derived cells such as endothelial cells (BECs), neurons, microglia, and astrocytes, can be found in the circulation. BECs are the most proximate cells to circulating blood, and are thus the most prone to be shed into the circulation. As a result, cBECs can serve as rich diagnostic markers, e.g., “footprints,” of brain disorders and disease, such as concussions and traumatic brain injury of various levels, when the clinical signs may be uncertain.

Brain-derived particles such as organelles or extracellular vesicles, e.g., microvesicles (MVs), exosomes, oncosomes, and apoptotic bodies can also be shed from brain tissue into the circulation.

Circulating Endothelial Cells (cECs) in Cardiovascular Disease

Ample data indicate that cECs are biomarkers of injury (Erdbruegger, Haubitz et al. 2006). In normal individuals, the cEC concentration is <3/ml (Solovey, Lin et al. 1997, Dignat-George and Sampol 2000, Ryder, O'Connell et al. 2016), and slightly increases with age (Strijbos, Rao et al. 2008). In cardiovascular diseases (e.g., coronary catheterization, acute myocardial ischemia, sickle cell disease, systemic vasculitis, thrombotic thrombocytopenic purpura), cEC concentration can rapidly increase by ˜2-20 fold (George, Brisson et al. 1992, Lefevre, George et al. 1993, Solovey, Lin et al. 1997, Mutin, Canavy et al. 1999, Dignat-George, Blann et al. 2000, Dignat-George and Sampol 2000, Woywodt 2003, Woywodt, Streiber et al. 2003, Quilici, Banzet et al. 2004, Blann, Woywodt et al. 2005, Blann and Pretorius 2006, Erdbruegger, Haubitz et al. 2006, Vargova, Toth-Zsamboki et al. 2008, Bonello, Harhouri et al. 2010, Damani, Bacconi et al. 2012), typically in the form of intact cells, anuclear carcasses, or sheets of multiple cells as large as 100 μm in size (Erdbruegger, Haubitz et al. 2006). Importantly, cEC concentration correlates with the severity of the pathological process, and is accompanied by a rise in markers of endothelial activation (e.g., vWF, ICAM-1, VCAM-1, E and P selectin). cEC morphology may reflect the primary insult (Dignat-George and Sampol 2000).

### Circulating Brain-Derived Cells in Neurological Disease

Isolated central nervous system (CNS) disorders can increase the concentration of circulating brain-derived cells, such as circulating endothelial cells (cEC), for example in primary CNS vasculitis (>50-fold) (Deb, Gerdes et al. 2013), and acute focal cerebral ischemia (up to 10-fold) (Bardy 1980, Wu, Liu et al. 2000, Freestone, Lip et al. 2005, Nadar, Lip et al. 2005, Gao, Liu et al. 2008, Woywodt, Gerdes et al. 2012, Deb, Gerdes et al. 2013). Assuming that ECs detected in these studies were BECs, these data suggest that BECs can be shed into the circulation in neurological diseases as well. Indeed, cBEC concentrations increase after generalized bicuculline- or kainate-induced seizures in piglets (Parfenova, Leffler et al. 2010), and in infants with seizures, asphyxia and intraventricular hemorrhage (Pourcyrous, Basuroy et al. 2015), providing further proof-of-concept for our proposal.

Microfluidics vs. Traditional Methods to Isolate and Quantify cECs

Previous studies to capture cBECs have used flow cytometry with FACS, which has significant caveats: (a) cBECs are ultra-rare (few cells/ml whole blood) (Goon, Boos et al. 2006), at or below the detection limit of flow cytometry (Woywodt, Gerdes et al. 2012); (b) FACS is labor intensive, expensive, time consuming, not available everywhere, especially not at the bedside; and (c) cross-reactivity of established antibodies with platelets, lymphocytes, cECs originating in other organs, and circulating endothelial progenitor cells (cEPC; immature cells originating in bone marrow) can confound FACS.

The new microfluidic technology described herein offers distinct advantages over FACS. High-fidelity negative depletion of circulating blood cells eliminates the vast majority of platelets, erythrocytes (red blood cells (RBCs)), and white blood cells (WBC). In this way, the microfluidic chip achieves unprecedented enrichment of cBECs, greatly increasing its sensitivity to capture even a single cell in 10⁹ blood cells. Captured cells in the product are then interrogated (i.e., ddPCR and immunolabeling) for a sensitive and specific molecular signature to distinguish the target cells. Moreover, because the blood is processed uniformly and physiologically, and the transit time is only 3 seconds, isolated cells are alive and healthy for accurate phenotypic characterization, in vitro (e.g., scRNAseq). In other implementations, positive selection can also be used.

Of course, cells such as BECs can be analyzed as described herein, e.g., using ddPCR, even if the cells have been isolated using known methods and systems of isolation other than the microfluidic methods and systems described herein.

### Impact Beyond Traumatic Brain Injury

The new systems and methods are not limited to diagnosing and monitoring TBI. The cerebrovascular bed is embedded deep in the brain tissue and thus not easily accessible. The new technology described herein can provide for the first time direct and easy access to brain-derived cells, such as BECs, neurons, microglia, and astrocytes, and brain-derived particles such as organelles or extracellular vesicles, e.g., microvesicles (MVs), exosomes, oncosomes, and apoptotic bodies, for molecular, physiological, and pharmacological characterization in a large array of acute or chronic neurologic, psychiatric, and even systemic diseases. BECs regulate vascular tone, WBC adhesion, hemostasis, and the blood-brain barrier (BBB), and respond to pathological states by changing their phenotype (e.g. expression of surface markers and BBB regulatory proteins). Therefore, besides the changes in the concentration of cBEC and other brain-derived cells and particles, changes in phenotype and gene expression can herald, for example, acute transient cerebral ischemic attacks, or chronic progressive small vessel disease and vascular dementia.

Moreover, the technology described herein can be adapted to detect and capture other circulating cell types originating from the brain (e.g., neurons, astrocytes, microglia, and pericytes), and even cell particles (e.g., extracellular vesicles such as exosomes and microparticles (Combes, Simon et al., 1999)). There has never been an attempt to seek and capture such brain-derived circulating cells or cell particles with the sensitivity and specificity afforded by microfluidics. Therefore, proposed technology may have impact far beyond the scope of this proposal, where circulating brain-derived cells can be utilized for research, diagnostic and therapeutic purposes, especially in diseases where a brain biopsy is the only diagnostic option.

Microfluidic Systems—the iChip

The ideal tool to capture cBECs should have high sensitivity and specificity, and be cheap, quick, and portable for the bedside (e.g., small clinics) and the field (e.g. ambulances, football, or battlefields). We developed a microfluidic chip (so-called “iChip”) for inertial focusing) to separate ultra-rare cells directly from whole blood by negative depletion ((Ozkumur, Shah et al. 2013, Fachin, Spuhler et al. 2017). The device is independent of preselected surface markers on target cells (i.e., target antigen-independent), which is a major advance in rare cell isolation. However, in some implementations, the system can also be used for positive selection of the target cells when specific preselected surface markers are known for the target cells.

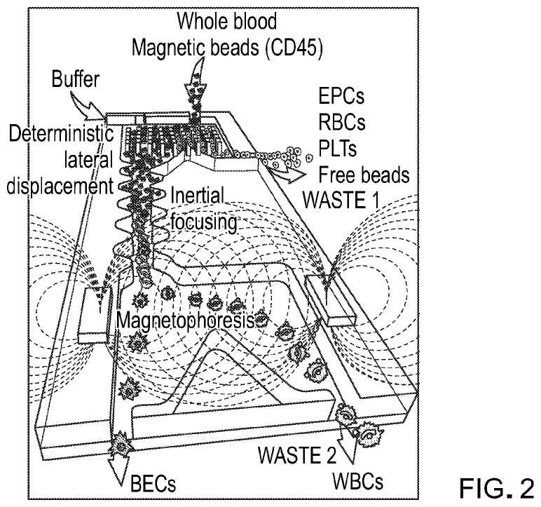

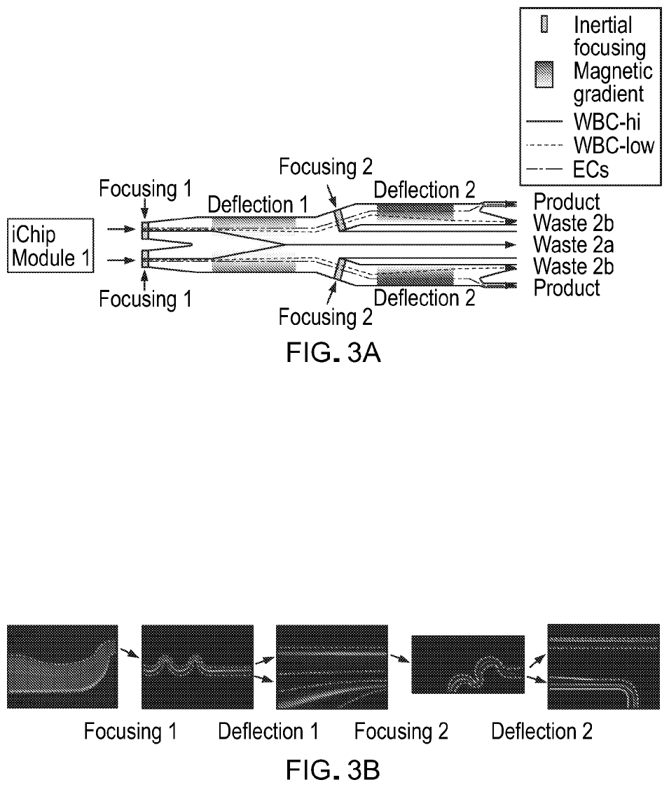

Key steps are illustrated in FIGS. 2 and 3, and are described as follows:

(1) Magnetic tagging of target cells or particles with magnetic beads that include binding agents that specifically bind to the target cells or particles. For example, in a negative depletion (i.e., negative selection) mode, the target cells can be WBCs, and they can be bound to the magnetic beads (e.g., 1, 2, 3, 4, or 5 microns in diameter) using antibodies conjugated, e.g., biotin-conjugated, to the surface of the beads, such as anti-WBC antibodies, e.g., anti-CD45, anti-CD16, and anti-CD66b antibodies. In other embodiments, a positive selection mode is used in which the target cells are the cells of interest, such as endothelial cells, which can be targeted using anti-CD31, anti-CD146, anti-VE-cadherin, anti-CD34, anti-SLCO1C1, anti-SLC22A8, anti-SLCO1A4, anti-CD133, anti-Tie2, anti-OCLN, anti-MFSD2A antibodies coated, i.e., conjugated on the surface of, magnetic beads. Antibodies to GFAP, FOX-3, and OLIG-2 would work to target other brain-derived cells including astrocytes, neurons, and oligodendrocytes. Similarly, other antibodies are known for specifically binding to certain brain-derived particles.

(2) Size-based separation (in a first module of the microfluidic chip), e.g., inertial exchanger or deterministic lateral displacement, for separation of small cells and particles (e.g., endothelial progenitor cells (EPCs), red blood cells (RBCs), and platelets) into Waste Outlet 1. This step is analogous to centrifugation or Ficoll gradient to prepare “buffy coat,” but with ultra-high precision.

(3) Inertial focusing (in a second module of the microfluidic chip) of WBCs and target cells, e.g., endothelial cells, in a microfluidic inertial focusing channel to align the cells into one or more streamlines within the flowing blood sample (analogous to flow cytometry, but without the sheath flow), to facilitate high-fidelity deflection into waste or product channels with minimal magnetic moment (FIGS. 3A (schematic) and 3B (microscope images)).

(4) Magnetophoresis (in a third module) of i) WBCs into Waste Outlet 2 (negative depletion mode), leaving highly purified, untouched, and viable target cells and particles, such as endothelial cells in the Product Outlet (similar to magnetic-activated cell sorting, but with extreme precision and sensitivity, without target cell injury), or ii) endothelial cells into Waste Outlet 2, to sort them with high purity and sensitivity (positive selection mode).

The microfluidic chip (iChip) can process large volumes of blood (40 ml/h) with unmatched throughput (20 million cells/sec), without losing target brain-derived cells and/or particles. The iChip, originally developed for circulating tumor cell detection, has been validated extensively to detect and separate even a single target cell in 1 ml whole blood. The present system is refined to capture brain-derived cells and/or particles, such as cBECs. The iChip is unique among other microfluidic methods for rare cell or particle isolation, because it yields the separated cells in a suspension amenable directly for subsequent imaging (hyperspectral fluorescent cell counting) or molecular analysis (ddPCR, scRNAseq) (Blann, Woywodt et al., 2005). The viability and functionality of the separated cells have been tested extensively.

Other microfluidic methods rely on laminar flow of cells through antibody-coated microposts or microvortices generated by herringbone-shaped grooves to direct cells toward antibody-coated surfaces, where cells are immobilized and not readily available for imaging or single-cell molecular characterization. Other commercially available or experimental approaches to rare-cell separation, such as magnetically-activated-cell-sorting (MACS), Ficoll-Paque or filtration techniques to separate WBCs from erythrocytes and other blood components, suffer from poor yield and purity and lack the rigorous and sensitive separation that is critical when the target cells are ultra-rare (e.g. 1-100/ml). The iChip overcomes these difficulties.

Use of Droplet Digital PCR (ddPCR) to Quantify Brain-Derived Cells and Particles

Studies in the cancer field demonstrated that the semi-quantitative RT-PCR analyses of ultra-rare circulating tumor cells have been inconsistent in part because of the relatively low sensitivity and specificity of RT-PCR when using whole blood. Indeed, ˜1 target cell/million is below the detection limit of RT-PCR for a non-abundant transcript. Even a very low background transcription of highly tissue-specific transcripts by abundant blood cells becomes a confounder when target cells are present at such vanishingly low numbers. The susceptibility of qRT-PCR to the inhibitory effects of large amounts of non-specific template also adds to the large variability and inconsistencies in rare cell detection. To overcome these challenges, quantitative ddPCR is used after the initial enrichment of cBECs under RNA-preserving conditions.

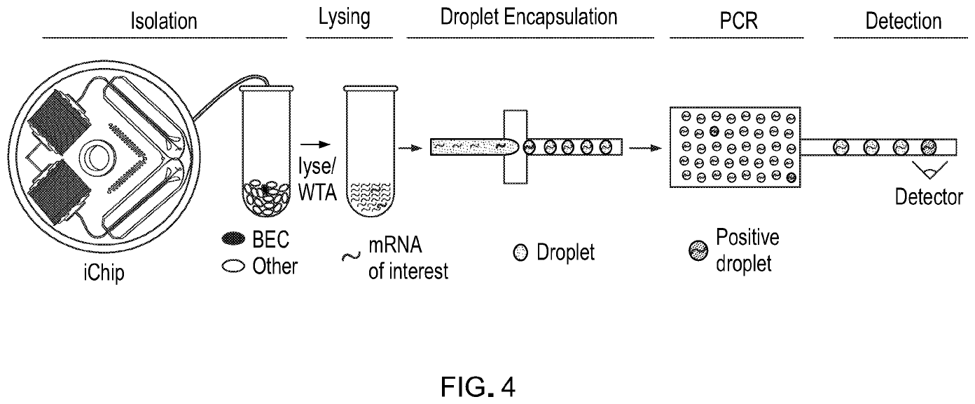

FIG. 4 shows a generic scheme for using ddPCR to analyze brain-derived cells or particles, such as endothelial cells, isolated in the microfluidic systems described herein to determine which, if any, of the isolated cells or particles, e.g., endothelial cells, originated from brain tissue. First, the cells, e.g., ECs, of which only a few may be BECs, are lysed and undergo WTA (Whole Transcriptome Amplification). Individual cells are encapsulated, e.g., using a system as described in, e.g., U.S. Pat. No. 9,068,181, which is incorporated herein by reference in its entirety.

Positive droplets are analyzed by ddPCR, which sequesters a small number of cDNA templates and PCR reaction reagents into aqueous droplets within an oil suspension, drastically increasing the effective concentration of the target transcript and allowing the differential expression of rare BEC-specific genes to be leveraged. Partitioning the entire cDNA sample into these droplets followed by high-cycle PCR to amplify each template of interest maximally creates a digital readout of the number of positive droplets as a measure of the prevalence of each transcript of interest. By tabulating the total number of positive and negative droplets, and assuming the transcripts of interest follow a Poisson-distribution when partitioning into droplets, the absolute number of transcripts in the sample can be imputed. ddPCR can quantify multiple lineage-specific transcripts that are absent from background and hence denote the presence of cBECs. See, e.g., PCT WO 2016/154600, which is incorporated herein by reference in its entirety, for a description of ddPCR.

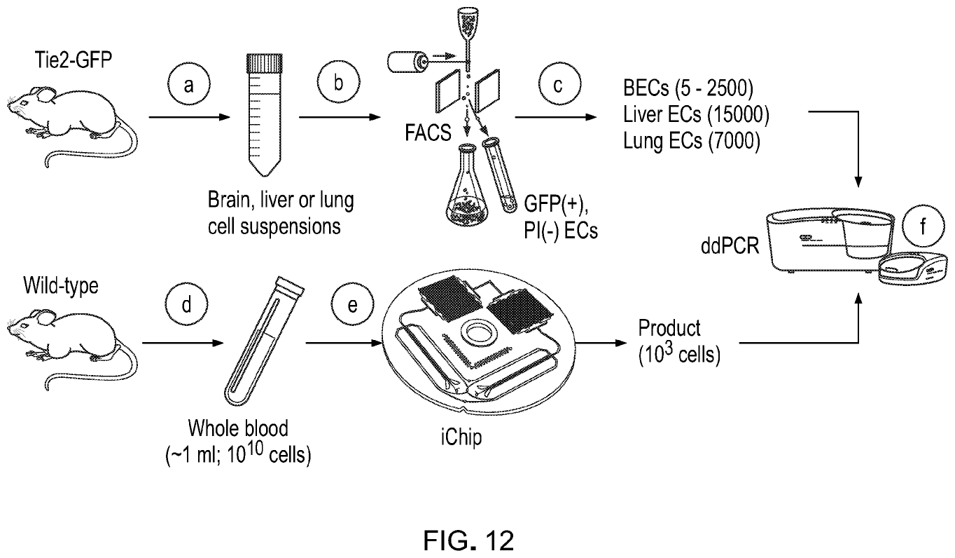

In experiments described below, Occludin and Prominin1 transcripts were found to be highly specific for BECs over lung and liver ECs, and absent in normal blood (FIG. 12). Additional BEC-specific transcripts in whole blood are described below as well.

### EXAMPLES

The invention is further described in the following examples, which do not limit the scope of the invention described in the claims.

### Example 1—iChip Captures BECs in Whole Blood Samples with ˜100% Efficiency

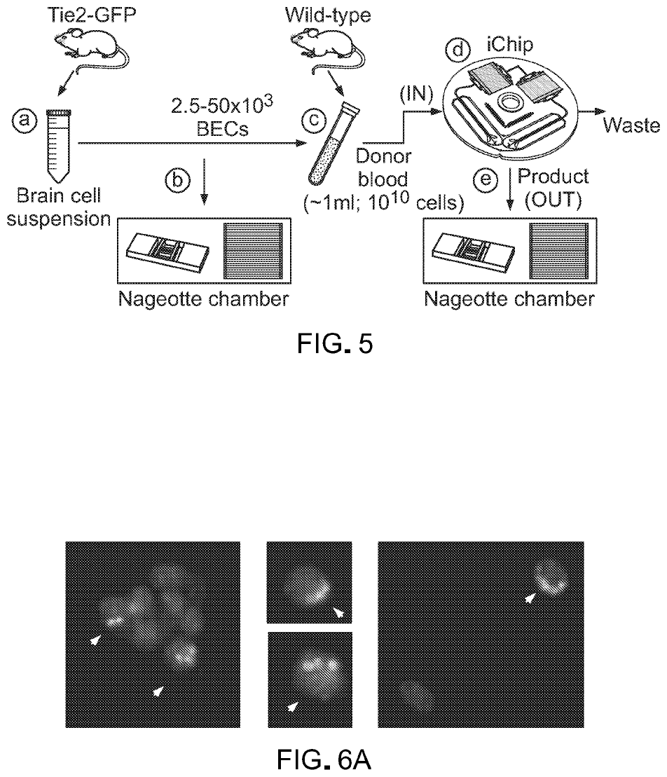

We first tested how well the latest iChip (i.e., without specific modifications targeting BECs) captures BECs in whole blood (FIG. 5). For step (a) we prepared brain cell suspensions from Tie2-GFP transgenic mice expressing green fluorescent protein (GFP) in endothelial cells under the direction of receptor tyrosine kinase, Tie2. These mice are currently in our breeding colonies. Suspensions were prepared as previously described ((Hickman, Allison et al. 2008, Hu, Ota et al. 2010, Metcalf and Griffin 2011). Dissociated cells were filtered through a 70 μM strainer. FACS showed that BECs make up ˜30% of live cells in the suspension (not shown). For step (b) we determined the BEC concentration in the suspension using a Nageotte cell counting chamber under wide-field fluorescence microscopy; Nageotte chambers are designed for rare cell studies with a volume larger than standard hemocytometers (100 μl active counting area).

For step (c), we obtained whole blood (˜1 ml, 10¹⁰ cells) from a wild-type (i.e. non-GFP) mouse, and spiked it with 2,500-50,000 fluorescent BECs using the brain cell suspension, in vitro.

For step (d) we ran the spiked sample through iChip, to obtain the product.

In step (e), we counted the GFP+ BECs in the product.

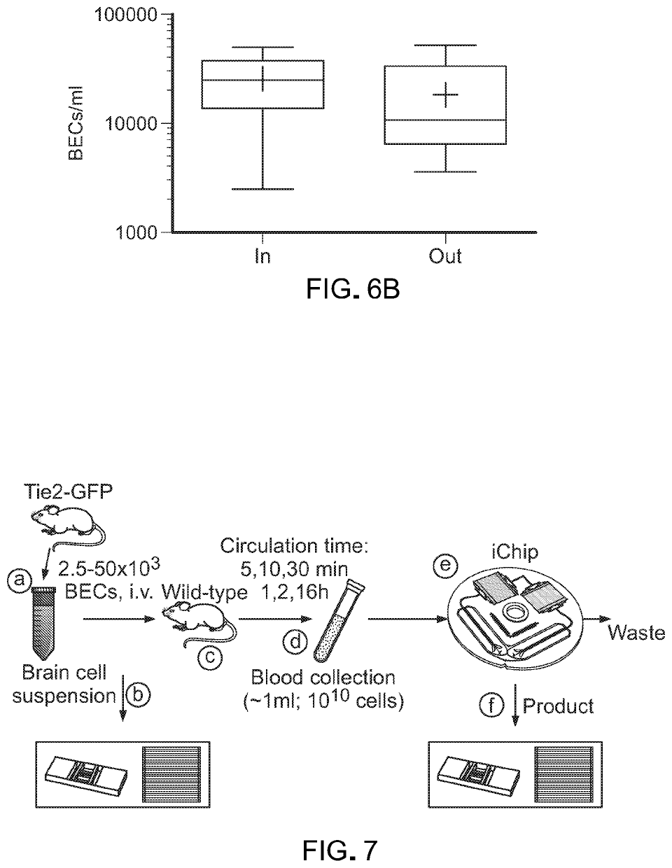

We found that 78±20% of all BECs spiked in the whole blood were captured in the product (n=5). FIG. 6A is a series of green fluorescent protein (GFP)+ BECs (arrowheads) in an iChip product. Non-GFP cells are also present (DAPI nuclear stain).

FIG. 6B is a graph of BEC counts in 1 ml whole blood (In) and in iChip product (Out). As shown in this graph, the iChip enriched the BECs from 1-20 BECs in ˜4 million blood cells to 1 BEC in ˜10 cells. We suspect that a subset of DAPI+ cells in the product were indeed other types of spiked brain cells (e.g., neurons, astrocytes) from the suspension; therefore, the denominator should be smaller in real life situations. These data show that iChip captures BECs in whole blood with little loss.

### Example 2—iChip Captures cBECs after 16 Hours of Circulating in Systemic Blood

We next examined our ability to detect cBECs after intravenous injection into recipient CD-1 mice (i.e., spiking the mouse with cell suspension). The steps of this example are illustrated in FIG. 7. In step (a) we prepared brain cell suspension from Tie2-GFP mice. In step (b) we determined the BEC in Nageotte chambers. In step (c) we injected 2.5, 25, or 75×10³ BECs in 100 to 300 μl of saline via the tail vein in a wild-type mouse, in vivo. In step (d), after allowing the cells to circulate 5 minutes to 16 hours (e.g., 5, 10, 30, 60, 120, or 960 minutes) in recipient mice, we collected blood (˜1 ml). In step (e) we processed the collected blood in an iChip. In step (f) we quantified cBECs.

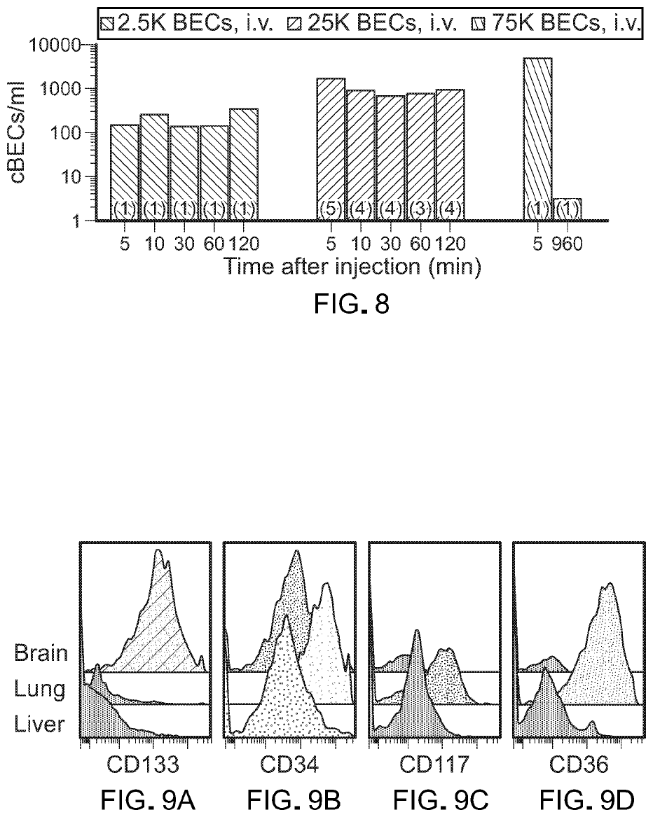

We detected cBECs in all cases, even after 960 min (16 hours) of circulation (see the bar graphs in FIG. 8, which are graphs of whole blood (including cBEC) captured in an iChip product after intravenous injection of three different dose levels (2.5K, 25K, and 75K) of fluorescent BECs, circulating between 5-960 min in recipient mice. Sample sizes (N) shown on each bar represent number of mice). More importantly, we were able to show a dose-response. As little as 2500 BECs injected via tail vein yielded robust cBEC concentration at 120 minutes. As predicted, numbers of cBECs captured in whole blood was much lower than the injected numbers (˜10%) presumably due to elimination of the exogenously introduced cells by, for example, the lungs. Nevertheless, the surviving cBEC counts remained steady over time. These data show that iChip can detect cBECs in whole blood samples with a dose-response relationship, and cBECs can circulate for many hours as a potential biomarker.

### Example 3—BEC Immunomarkers Reliably Differentiate BECs from Other ECs

After enriching BECs in whole blood by the iChip, we have to differentiate them from cECs originating from other organs. To this end, we prepared cell suspensions from brain, lung, and liver (Hickman, Allison et al. 2008, Hu, Ota et al. 2010, Metcalf and Griffin 2011), and labeled the cells with anti-CD31 (BV605), anti-CD133 (PE), anti-CD117 (AF488), anti-mouse CD34 (AF647), and anti-mouse CD36 (AF488). We then performed multi-channel flow cytometry/FACS, and compared the expression of CD133, CD34, CD117 and CD36 by CD31+ ECs from brain, lung, and liver (n=1 each). We found BECs to selectively express CD133 (Prominin1) at very high levels, and very little CD117 or CD36 (see FIGS. 9A-9D, which is a series of graphs of multichannel flow cytometry/FACS demonstration of expression of four surface markers in CD31+/PI− ECs from brain, lung, and liver). These data show that the CD31/CD133 combination is a good marker for an antigenic signature for BECs.

### Example 4—Bulk RNAseq Identifies Transcripts Specific for BECs Over ECs from Other Organs

Although several studies in the literature provided insight into molecular signature specific to brain endothelial cells, we aimed to validate these findings, expand the molecular signature tailored more towards our question at hand: What are the transcripts that will provide “BEC burden” in blood samples? With this goal we used Tie2-GFP mice, isolated 600 liver, lung, brain ECs (n=4 for brain, lung and n=2 for liver) using FACS and collected cells remaining in iChip product from blood samples. The data provided quantification of 45,000 transcripts with an approximate coverage of 40% of the genome. 508 transcripts were detected as candidate markers of brain endothelial cells with the following conditions: 1) higher reads than the top 5^{th} quantile in BECs, 2) lower or equal to a sum of single read in all other tissues.

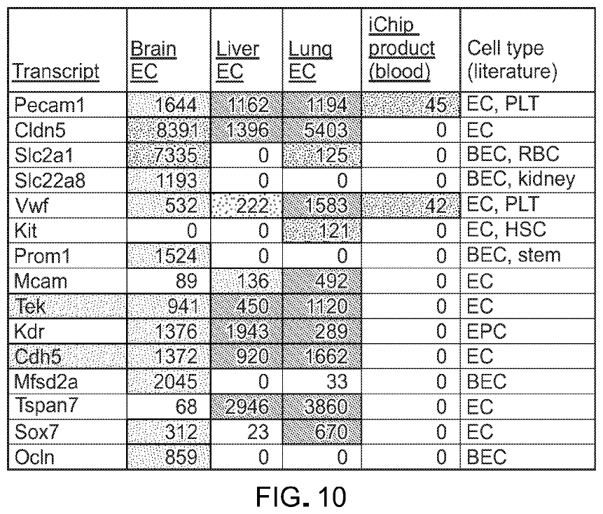

As shown in the table in FIG. 10, focusing on the transcripts related to commonly used markers to identify endothelial cells as well as known blood brain barrier proteins, bulk RNAseq revealed OCLN and PROM1 are highly expressed in BECs, but are not detected in blood, liver, or lung ECs. Another useful transcript is SLC22A8.

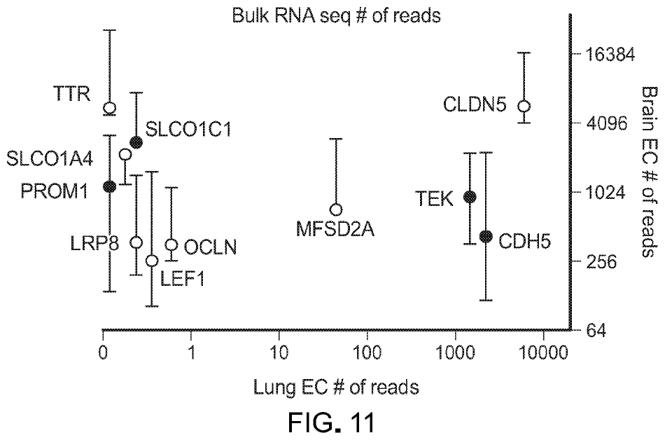

In addition, as illustrated in FIG. 11 we identified additional candidate transcripts that might provide high sensitivity and specificity for BECs, including TTR, SLCO1A4, LRP8, LEF1 in addition to SLCO1C1, PROM1, OCLN. Also, CLDN5, TEK, CDH5 provides sensitive detection of brain, lung and other endothelial cells.

### Example 5—ddPCR Differentiates BECs in iChip Product with High Sensitivity and Specificity

Counting BECs using immunomarkers is relatively expensive and time consuming, and leads to higher cell losses during processing. Moreover, there is no single “gold-standard” immunomarker, and presence of non-specific antibody binding can be an obstacle. In the present studies, we employed ddPCR as a complementary approach to identify and quantify BECs. The steps of this example are illustrated in FIG. 12. First, in step (a) we prepared brain, liver, and lung cell suspensions from Tie2-GFP mice. In step (b) we sorted GFP+/PI− live ECs from brain, liver, and lung suspension using FACS. In step (c) we prepared suspensions with known numbers of brain, liver, or lung ECs. In parallel, in step (d) we collected ˜1 ml whole blood from naïve wild-type mice to serve as negative control, and in step (e) processed the blood through the iChip to obtain the product. In step (0 we then performed ddPCR on these samples to measure transcript numbers for Occludin, a tight junction protein specifically enriched in blood-brain barrier (Daneman, Zhou et al. 2010), and Prominin1 (i.e. CD133), highly expressed in BECs (Nolan, Ginsberg et al. 2013).

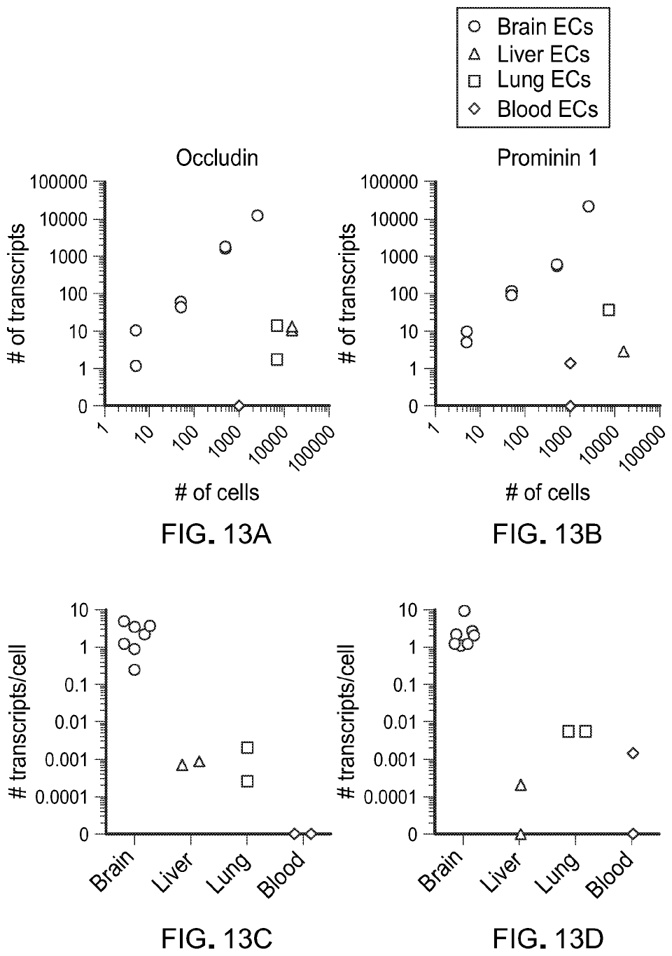

FIGS. 13A-13D is a series of four graphs that show ddPCR transcript counts in brain, liver and lung ECs, and naïve whole blood in an iChip product. As illustrated in these four graphs, we found that both Occludin and Prominin1 transcripts provided ultra-high sensitivity, detecting even 5 BECs (n=2 each sample; FIGS. 13A and 13B). Moreover, both transcripts were highly specific for BECs, which expressed 2000 and 3000 times higher Occludin (2.3±0.6 transcripts/BEC), and 500 and 25000 times higher Prominin1 (2.6±1.0 transcripts/BEC), compared with liver and lung ECs, respectively (see FIGS. 13C and 13D). Last but not the least, Occludin and Prominin1 transcripts were nearly undetectable in whole blood iChip product obtained from naïve mice, suggesting that the background signal is close to zero. These data show that ddPCR significantly enhances our sensitivity and specificity, and scales down our BEC detection limit.

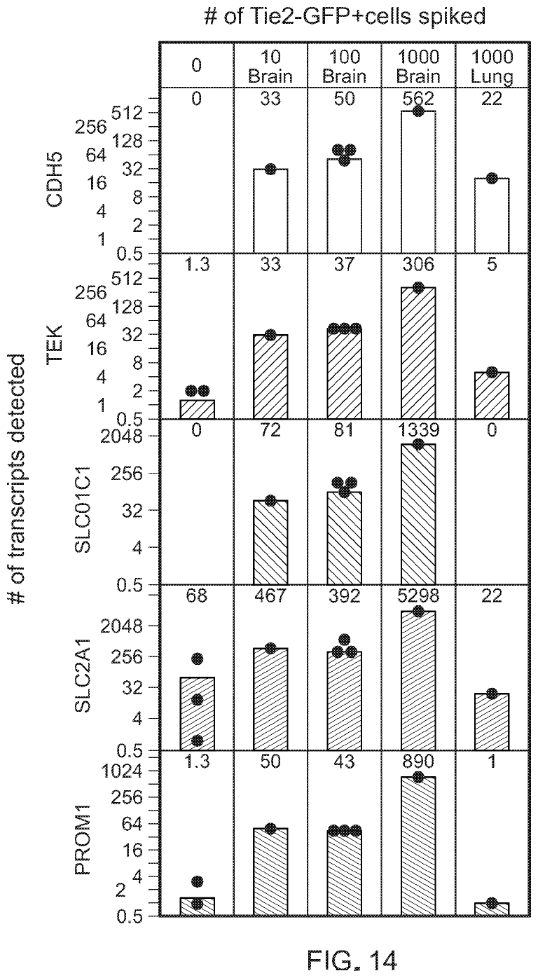

We also performed a similar experiment with a cocktail of 5 ddPCR probes (SLC22A8, SLCO1C1, TEK, PROM1, CDH5) where mouse brain suspension containing ˜10, 100, or 1,000 Tie2GFP+ endothelial cells, ˜1,000 lung Tie2GFP+ endothelial cells, or no cells (0) were spiked into mouse blood samples. We depleted blood cells using the iChip and performed ddPCR on the sorted product. The Y axis shows number of droplets detected for specific transcripts and average transcripts detected is indicated on the top of the plot (n=3 for 100 and 0 brain EC spike experiment, n=1 for others, each dot represents a spiked mouse blood sample from one animal). The results of this experiment are illustrated in FIG. 14, which shows we obtained sensitive and specific detection of BECs from the iChip product.

### Example 6—CHI Acutely Increases cECs

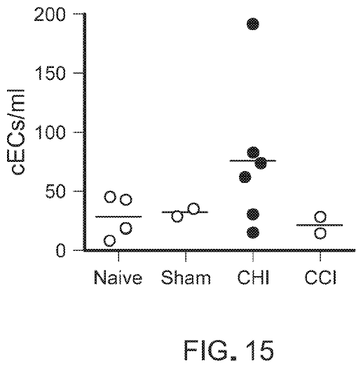

To test whether concussion leads to BEC shedding, we induced a severe CHI in Tie2-GFP mice, collected whole blood (˜1 ml) 5 minutes later, and processed it through the iChip, as described in FIGS. 7d-f above. As illustrated in the graph of FIG. 15, we found a more than two-fold increase in cECs after concussion (red dots/CHI) compared with naïve or sham-injured mice (yellow dots). Interestingly, contusion using an open skull, controlled cortical impact (CCI) model (i.e., stationary head), did not appear to increase cBECs, suggesting that acceleration-deceleration may be critical for BEC shedding, and thus specific for concussive injury (yellow dots/CCI).

Since there is a fair amount of cell loss during the counting procedure (e.g., dead space in Nageotte chamber), we anticipate even higher signal-to-noise ratios over background counts using ddPCR.

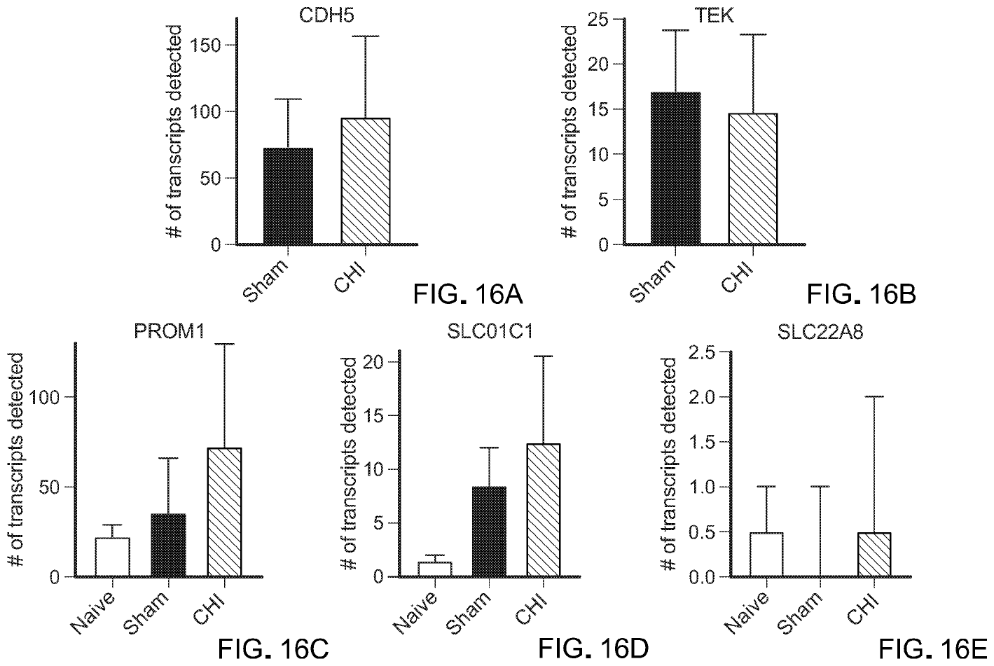

To specifically quantify cBECs, we performed ddPCR mouse CHI or musculoskeletal injury (sham), obtaining blood samples from these mice (˜1 mL), red blood cell lysis of blood samples and following ddPCR protocol as shown in FIG. 4. ddPCR probes identified as specific to BECs (PROM1, SLC01C1, SLC22A8, see FIGS. 16C-16E) and all endothelial cells (TEK, CDH5, see FIGS. 16A and 16B) were used to quantify their levels. Naïve mice were not injured (n=2 for naïve, n=10 for sham, n=14 for TBI). We indeed detected a significant increase of SLCO1C1 transcripts following CHI compared to naïve mice (p<0.05) and to sham injury (p<0.1). In addition, the median number of PROM1 transcripts was trending higher after CHI compared to sham injury and significantly higher than naïve mice (p<0.1) (FIG. 16C). These results suggest a specific increase of cBECs following CHI, a model of traumatic brain injury.

### Example 7—Ischemia Acutely Increases cBECs

To test whether stroke leads to BEC shedding, we induced a transient occlusion of the middle cerebral artery in wild type mice as an experimental model of stroke, collected whole blood (˜1 ml) 5 minutes later, and processed the blood through a ddPCR protocol as indicated in FIG. 4.

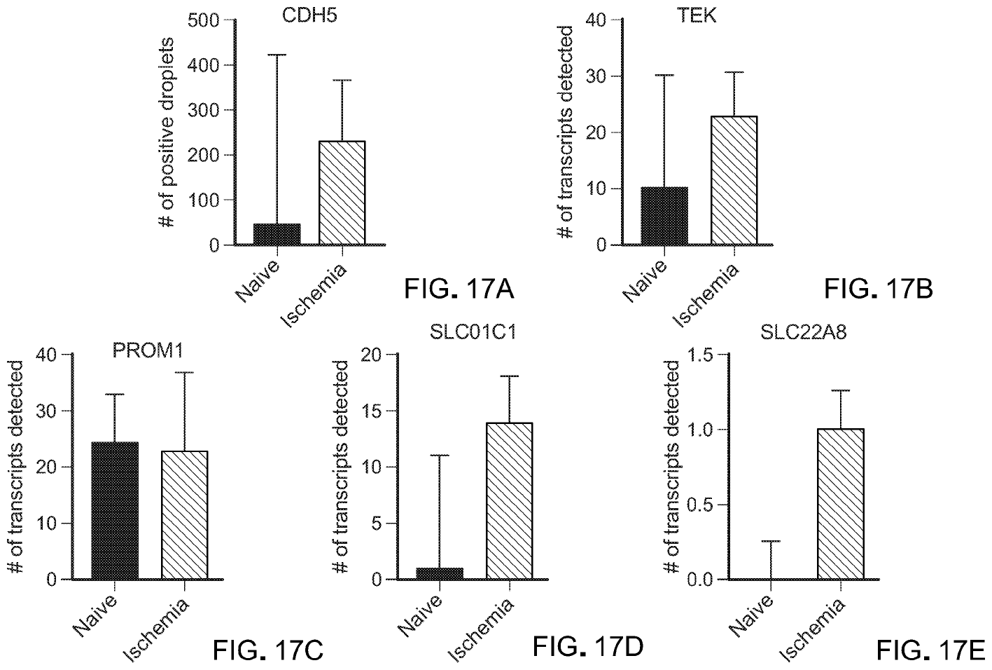

The results are shown in FIGS. 17A-17E, which is a series of graphs for this experiment in which we used the same set of probes as in Example 6 to quantify BECs (PROM1, SLC01C1, SLC22A8, FIGS. 17C-17E) and all endothelial cells (TEK, CDH5, FIGS. 17A and 17B) in blood samples. Naïve mice were not injured (n=6 for each condition). We detected a significant increase in SLCO1C1 (from a median of 1 to 14 transcripts detected) suggesting an increase in cBECs. In addition, medians of CDH5, SLC22A8, and TEK were trending higher.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Bardy, M. (1980). “Demonstration of pathological circulating endothelial cells in patients with amaurosis fugax.” Trans Ophthalmol Soc U K 100(3): 385-387.
Blann, A. D. and A. Pretorius (2006). “Circulating endothelial cells and endothelial progenitor cells: two sides of the same coin, or two different coins?” Atherosclerosis 188(1): 12-18.
Blann, A. D., A. Woywodt, F. Bertolini, T. M. Bull, J. P. Buyon, R. M. Clancy, M. Haubitz, R. P. Hebbel, G. Y. Lip, P. Mancuso, J. Sampol, A. Solovey and F. Dignat-George (2005). “Circulating endothelial cells. Biomarker of vascular disease.” Thromb Haemost 93(2): 228-235.
Bonello, L., K. Harhouri, F. Sabatier, L. Camoin-Jau, L. Arnaud, K. Baumstarck-Barrau, O. Ait-Mokhtar, F. Roubille, C. Piot, N. Lesavre, F. Paganelli and F. Dignat-George (2010). “Level of adenosine diphosphate receptor P2Y12 blockade during percutaneous coronary intervention predicts the extent of endothelial injury, assessed by circulating endothelial cell measurement.” J Am Coll Cardiol 56(13): 1024-1031.
Buckley, E. M., B. F. Miller, J. M. Golinski, H. Sadeghian, L. M. McAllister, M. Vangel, C. Ayata, W. P. Meehan, 3rd, M. A. Franceschini and M. J. Whalen (2015). “Decreased microvascular cerebral blood flow assessed by diffuse correlation spectroscopy after repetitive concussions in mice.” J Cereb Blood Flow Metab 35(12): 1995-2000.
Combes, V., A. C. Simon, G. E. Grau, D. Arnoux, L. Camoin, F. Sabatier, M. Mutin, M. Sanmarco, J. Sampol and F. Dignat-George (1999). “In vitro generation of endothelial microparticles and possible prothrombotic activity in patients with lupus anticoagulant.” J Clin Invest 104(1): 93-102.
Damani, S., A. Bacconi, O. Libiger, A. H. Chourasia, R. Serry, R. Gollapudi, R. Goldberg, K. Rapeport, S. Haaser, S. Topol, S. Knowlton, K. Bethel, P. Kuhn, M. Wood, B. Carragher, N. J. Schork, J. Jiang, C. Rao, M. Connelly, V. M. Fowler and E. J. Topol (2012). “Characterization of circulating endothelial cells in acute myocardial infarction.” Sci Transl Med 4(126): 126ra133.
Daneman, R., L. Zhou, D. Agalliu, J. D. Cahoy, A. Kaushal and B. A. Barres (2010). “The mouse blood-brain barrier transcriptome: a new resource for understanding the development and function of brain endothelial cells.” PLoS One 5(10): e13741.
Deb, M., S. Gerdes, M. Heeren, J. Lambrecht, H. Worthmann, A. Goldbecker, A. B. Tryc, S. Lovric, W. Schulz-Schaeffer, A. Brandis, R. Dengler, K. Weissenborn and M. Haubitz (2013). “Circulating endothelial cells as potential diagnostic biomarkers in primary central nervous system vasculitis.” J Neurol Neurosurg Psychiatry 84(7): 732-734.
Dignat-George, F., A. Blann and J. Sampol (2000). “Circulating endothelial cells in acute coronary syndromes.” Blood 95(2): 728.
Dignat-George, F. and J. Sampol (2000). “Circulating endothelial cells in vascular disorders: new insights into an old concept.” Eur J Haematol 65(4): 215-220.
Erdbruegger, U., M. Haubitz and A. Woywodt (2006). “Circulating endothelial cells: a novel marker of endothelial damage.” Clin Chim Acta 373(1-2): 17-26.
Fachin, F., P. Spuhler, J. M. Martel-Foley, J. F. Edd, T. A. Barber, J. Walsh, M. Karabacak, V. Pai, M. Yu, K. Smith, H. Hwang, J. Yang, S. Shah, R. Yarmush, L. V. Sequist, S. L. Stott, S. Maheswaran, D. A. Haber, R. Kapur and M. Toner (2017). “Monolithic Chip for High-throughput Blood Cell Depletion to Sort Rare Circulating Tumor Cells.” Sci Rep 7(1): 10936.
Freestone, B., G. Y. Lip, A. Y. Chong, S. Nadar, K. W. Lee and A. D. Blann (2005). “Circulating endothelial cells in atrial fibrillation with and without acute cardiovascular disease.” Thromb Haemost 94(4): 702-706.
Gao, Y., C. Liu, X. Zhang, J. Gao and C. Yang (2008). “Circulating endothelial cells as potential markers of atherosclerosis.” Can J Neurol Sci 35(5): 638-642.
George, F., C. Brisson, P. Poncelet, J. C. Laurent, O. Massot, D. Arnoux, P. Ambrosi, C. Klein-Soyer, J. P. Cazenave and J. Sampol (1992). “Rapid isolation of human endothelial cells from whole blood using S-Endol monoclonal antibody coupled to immuno-magnetic beads: demonstration of endothelial injury after angioplasty.” Thromb Haemost 67(1): 147-153.
Goon, P. K., C. J. Boos, P. S. Stonelake, A. D. Blann and G. Y. Lip (2006). “Detection and quantification of mature circulating endothelial cells using flow cytometry and immunomagnetic beads: a methodological comparison.” Thromb Haemost 96(1): 45-52.
Helmick, K. M., C. A. Spells, S. Z. Malik, C. A. Davies, D. W. Marion and S. R. Hinds (2015). “Traumatic brain injury in the US military: epidemiology and key clinical and research programs.” Brain Imaging Behav 9(3): 358-366.
Hickman, S. E., E. K. Allison and J. El Khoury (2008). “Microglial dysfunction and defective beta-amyloid clearance pathways in aging Alzheimer's disease mice.” J Neurosci 28(33): 8354-8360.
Hu, Y., N. Ota, I. Peng, C. J. Refino, D. M. Danilenko, P. Caplazi and W. Ouyang (2010). “IL-17RC is required for IL-17A- and IL-17F-dependent signaling and the pathogenesis of experimental autoimmune encephalomyelitis.” J Immunol 184(8): 4307-4316.
Kalish, B. T. and M. J. Whalen (2016). “Weight Drop Models in Traumatic Brain Injury.” Methods Mol Biol 1462: 193-209.
Khuman, J., W. P. Meehan, 3rd, X. Zhu, J. Qiu, U. Hoffmann, J. Zhang, E. Giovannone, E. H. Lo and M. J. Whalen (2011). “Tumor necrosis factor alpha and Fas receptor contribute to cognitive deficits independent of cell death after concussive traumatic brain injury in mice.” J Cereb Blood Flow Metab 31(2): 778-789.
Kondo, A., K. Shahpasand, R. Mannix, J. Qiu, J. Moncaster, C. H. Chen, Y. Yao, Y. M. Lin, J. A. Driver, Y. Sun, S. Wei, M. L. Luo, O. Albayram, P. Huang, A. Rotenberg, A. Ryo, L. E. Goldstein, A. Pascual-Leone, A. C. McKee, W. Meehan, X. Z. Zhou and K. P. Lu (2015). “Antibody against early driver of neurodegeneration cis P-tau blocks brain injury and tauopathy.” Nature 523(7561): 431-436.
Langlois, J. A., W. Rutland-Brown and M. M. Wald (2006). “The epidemiology and impact of traumatic brain injury: a brief overview.” J Head Trauma Rehabil 21(5): 375-378.
Lefevre, P., F. George, J. M. Durand and J. Sampol (1993). “Detection of circulating endothelial cells in thrombotic thrombocytopenic purpura.” Thromb Haemost 69(5): 522.
Mannix, R., W. P. Meehan, J. Mandeville, P. E. Grant, T. Gray, J. Berglass, J. Zhang, J. Bryant, S. Rezaie, J. Y. Chung, N. V. Peters, C. Lee, L. W. Tien, D. L. Kaplan, M. Feany and M. Whalen (2013). “Clinical correlates in an experimental model of repetitive mild brain injury.” Ann Neurol 74(1): 65-75.
Meehan, W. P., 3rd, J. Zhang, R. Mannix and M. J. Whalen (2012). “Increasing recovery time between injuries improves cognitive outcome after repetitive mild concussive brain injuries in mice.” Neurosurgery 71(4): 885-891.
Metcalf, T. U. and D. E. Griffin (2011). “Alphavirus-induced encephalomyelitis: antibody-secreting cells and viral clearance from the nervous system.” J Virol 85(21): 11490-11501.
Mutin, M., I. Canavy, A. Blann, M. Bory, J. Sampol and F. Dignat-George (1999). “Direct evidence of endothelial injury in acute myocardial infarction and unstable angina by demonstration of circulating endothelial cells.” Blood 93(9): 2951-2958.
Nadar, S. K., G. Y. Lip, K. W. Lee and A. D. Blann (2005). “Circulating endothelial cells in acute ischaemic stroke.” Thromb Haemost 94(4): 707-712.
Nolan, D. J., M. Ginsberg, E. Israely, B. Palikuqi, M. G. Poulos, D. James, B. S. Ding, W. Schachterle, Y. Liu, Z. Rosenwaks, J. M. Butler, J. Xiang, A. Rafii, K. Shido, S. Y. Rabbany, O. Elemento and S. Rafii (2013). “Molecular signatures of tissue-specific microvascular endothelial cell heterogeneity in organ maintenance and regeneration.” Dev Cell 26(2): 204-219.
Ozkumur, E., A. M. Shah, J. C. Ciciliano, B. L. Emmink, D. T. Miyamoto, E. Brachtel, M. Yu, P. I. Chen, B. Morgan, J. Trautwein, A. Kimura, S. Sengupta, S. L. Stott, N. M. Karabacak, T. A. Barber, J. R. Walsh, K. Smith, P. S. Spuhler, J. P. Sullivan, R. J. Lee, D. T. Ting, X. Luo, A. T. Shaw, A. Bardia, L. V. Sequist, D. N. Louis, S. Maheswaran, R. Kapur, D. A. Haber and M. Toner (2013). “Inertial focusing for tumor antigen-dependent and -independent sorting of rare circulating tumor cells.” Sci Transl Med 5(179): 179ra147.
Parfenova, H., C. W. Leffler, D. Tcheranova, S. Basuroy and A. Zimmermann (2010). “Epileptic seizures increase circulating endothelial cells in peripheral blood as early indicators of cerebral vascular damage.” Am J Physiol Heart Circ Physiol 298(6): H1687-1698.
Pourcyrous, M., S. Basuroy, D. Tcheranova, K. L. Arheart, M. T. Elabiad, C. W. Leffler and H. Parfenova (2015). “Brain-derived circulating endothelial cells in peripheral blood of newborn infants with seizures: a potential biomarker for cerebrovascular injury.” Physiol Rep 3(3).
Quilici, J., N. Banzet, P. Paule, J. B. Meynard, M. Mutin, J. L. Bonnet, P. Ambrosi, J. Sampol and F. Dignat-George (2004). “Circulating endothelial cell count as a diagnostic marker for non-ST-elevation acute coronary syndromes.” Circulation 110(12): 1586-1591.
Ryder, J. R., M. J. O'Connell, K. D. Rudser, C. K. Fox, A. N. Solovey, R. P. Hebbel and A. S. Kelly (2016). “Reproducibility of circulating endothelial cell enumeration and activation in children and adolescents.” Biomark Med 10(5): 463-471.
Solovey, A., Y. Lin, P. Browne, S. Choong, E. Wayner and R. P. Hebbel (1997). “Circulating activated endothelial cells in sickle cell anemia.” N Engl J Med 337(22): 1584-1590.
Strijbos, M. H., C. Rao, P. I. Schmitz, J. Kraan, C. H. Lamers, S. Sleijfer, L. W. Terstappen and J. W. Gratama (2008). “Correlation between circulating endothelial cell counts and plasma thrombomodulin levels as markers for endothelial damage.” Thromb Haemost 100(4): 642-647.
Vargova, K., E. Toth-Zsamboki, B. J. Beres, J. Bencze, G. Kerecsen, P. Gulacsi-Bardos, R. G. Kiss and I. Preda (2008). “Circulating endothelial cell count, plasma vWF and soluble ICAM-1 levels following primary or elective percutaneous coronary intervention.” Atherosclerosis 198(2): 366-372.
Woywodt, A. (2003). “Circulating endothelial cells in vasculitis and transplantation.” Pathophysiol Haemost Thromb 33(5-6): 500-502.
Woywodt, A., S. Gerdes, B. Ahl, U. Erdbruegger, M. Haubitz and K. Weissenborn (2012). “Circulating endothelial cells and stroke: influence of stroke subtypes and changes during the course of disease.” J Stroke Cerebrovasc Dis 21(6): 452-458.
Woywodt, A., F. Streiber, K. de Groot, H. Regelsberger, H. Haller and M. Haubitz (2003). “Circulating endothelial cells as markers for ANCA-associated small-vessel vasculitis.” Lancet 361(9353): 206-210.
Wu, S., S. Liu and R. Zhao (2000). “[Effect of longshoudan on serum tumor necrosis factor and circulating endothelial cell levels in acute cerebral infarction patients].” Zhongguo Zhong Xi Yi Jie He Za Zhi 20(2): 91-93.
Zhu, X., J. Park, J. Golinski, J. Qiu, J. Khuman, C. C. Lee, E. H. Lo, A. Degterev and M. J. Whalen (2014). “Role of Akt and mammalian target of rapamycin in functional outcome after concussive brain injury in mice.” J Cereb Blood Flow Metab 34(9): 1531-1539.

1. A method of analyzing brain-derived cells or particles from a blood sample from a subject, the method comprising
obtaining a blood sample from the subject;
mixing the blood sample with magnetic beads comprising a binding agent that specifically binds to white blood cells (WBCs) and not to the other cells or particles, for a time and under conditions sufficient for the binding agent to bind to the WBCs;
flowing the blood sample through a first module comprising a microfluidic size-based separation system configured to direct small cells and particles such as red blood cells (RBCs) and platelets in the blood sample to a first waste outlet and to direct the remaining blood sample to a second module comprising an inertial focusing channel;
flowing the remaining blood sample through the second module at a flow rate and for a distance sufficient to cause cells and/or particles in the remaining blood sample to align in one or more streamlines within the remaining blood sample flowing in the inertial focusing channel;
flowing the remaining blood sample with the cells and/or particles aligned in one or more streamlines through a third module comprising a magnetophoresis system for a time and distance sufficient to separate WBCs bound to magnetic beads from cells and particles not bound to magnetic beads, and flowing the WBCs into a second waste outlet and flowing other cells and particles to a product outlet;
obtaining cells or particles from the product outlet and determining which of the cells or particles originate in brain tissue; and
analyzing the brain-derived cells or particles.

2. A method of analyzing brain-derived cells or particles from a blood sample from a subject, the method comprising
obtaining a blood sample from the subject;
mixing the blood sample with magnetic beads comprising a binding agent that specifically binds to one or more specific types of cells or specific types of particles and not to white blood cells (WBCs), for a time and under conditions sufficient for the binding agent to bind to the brain-derived cells or particles;
flowing the blood sample through a first module comprising a microfluidic size-based separation system configured to direct small cells and particles such as red blood cells (RBCs) and platelets in the blood sample to a first waste outlet and to direct the remaining blood sample to a second module comprising an inertial focusing channel;
flowing the remaining blood sample through the second module at a flow rate and for a distance sufficient to cause cells and/or particles in the remaining blood sample to align in one or more streamlines within the remaining blood sample flowing in the inertial focusing channel;
flowing the remaining blood sample with the cells and/or particles aligned in one or more streamlines through a third module comprising a magnetophoresis system for a time and distance sufficient to separate the specific types of cells or particles bound to magnetic beads from WBCs cells, other cells, and particles not bound to magnetic beads, and flowing the WBCs other cells, and particles not bound to magnetic beads into a second waste outlet and flowing the bound cells or particles to a product outlet;
obtaining bound cells or particles from the product outlet and determining which of the bound cells or particles originate in brain tissue; and
analyzing the brain-derived cells or particles.

3. The method of claim 1, wherein the brain-derived cells comprise one or more of brain-derived endothelial cells (BECs), neurons, microglia, and astrocytes.

4. The method of claim 1, wherein the brain-derived particles comprise organelles or extracellular vesicles.

5. The method of claim 4, wherein the brain-derived extracellular vesicles comprise one or more of microvesicles (MVs), exosomes, oncosomes, and apoptotic bodies.

6. The method of claim 1, wherein the first module comprises an inertial exchanger configured to direct small cells such as red blood cells and platelets and particles in the blood sample to a first waste outlet and to direct the remaining blood sample to the second module.

7. The method of claim 1, wherein the first module comprises a deterministic lateral displacement array of microposts in a channel, wherein the array of microposts is configured to direct small cells such as red blood cells and platelets and particles in the blood sample to a first waste outlet and to direct the remaining blood sample to the second module.

8. The method of claim 1, wherein determining whether a cell or particle originated in brain tissue comprises analyzing the cell or particle using droplet digital PCR, an immunoassay, or both.

9. The method of claim 1, wherein determining whether a cell or particle originated in brain tissue comprises analyzing the cell or particle using detection of antigens unique to brain-derived cells or particles via fluorescently conjugated antibodies.

10. The method of claim 1, wherein determining whether a cell or particle originated in brain tissue comprises analyzing the cell or particle using brain-specific genes, transcripts, or proteins for differentiating brain-derived cells or particles from cells or particles of non-cerebral origin.

11. The method of claim 1, wherein determining whether a cell or particle originated in brain tissue comprises analyzing the cell or particle using single-cell RNA sequencing.

12. The method of claim 2, wherein the brain-derived cells comprise one or more of brain-derived endothelial cells (BECs), neurons, microglia, and astrocytes.

13. The method of claim 2, wherein the brain-derived particles comprise extracellular vesicles.

14. The method of claim 10, wherein the brain-specific genes comprise occludin and promininl, and wherein transcripts of these genes are used to detect brain-derived cells comprising brain-derived endothelial cells (BECs).

15. The method of claim 1, wherein the subject has a brain disorder selected from the group consisting of mild, moderate, or severe traumatic brain injury, vascular brain injury (selected from the group consisting of primary CNS vasculitis, acute focal cerebral ischemia, and small vessel disease), and neurodegenerative disease (selected from the group consisting of Alzheimer's disease, Parkinson's disease, and amyotrophic lateral sclerosis).

16. The method of claim 1, further comprising detecting a quantity of the brain-derived cells or particles, a quality of the brain-derived cells or particles, or both, for detecting a specific type of brain disorder or damage to the blood brain barrier.

17. The method of claim 1, wherein the magnetic beads specifically bind to WBCs and not to endothelial cells.

18. The method of claim 2, wherein the magnetic beads specifically bind to endothelial cells and not to WBCs.

19. The method of claim 1, further comprising separating the brain-derived cells, or brain-derived particles, from other cells and/or particles in the blood sample to isolate the brain-derived cells or particles.

20. A system for analyzing and/or isolating brain-derived cells or particles, from a blood sample from a subject, the system comprising
a mixer for combining the blood sample with magnetic beads comprising a binding agent that specifically binds to either
(i) brain-derived cells or particles and not to white blood cells (WBCs), or
(ii) WBCs and not to other cells or particles,
for a time and under conditions sufficient for the binding agent to bind;
a first module comprising a microfluidic size-based separation system configured to direct small cells and particles such as red blood cells and platelets in the blood sample to a first waste outlet;
a second module comprising an inertial focusing channel, wherein the remaining blood sample is controlled to flow through the second module at a flow rate and for a distance sufficient to cause cells and particles in the remaining blood sample to align in one or more streamlines within the remaining blood sample flowing in the inertial focusing channel;
a third module comprising a magnetophoresis system configured to separate cells or particles bound to magnetic beads from cells and particles not bound to magnetic beads, thus separating bound cells or particles from unbound cells and/or particles and flowing the WBCs into a second waste outlet and flowing unbound cells and/or particles to a product outlet; and
a fourth module comprising a cell or particle analyzer configured to determine which of the cells or particles originate in brain tissue and, optionally, to separate cells or particles originating in brain tissue from cells or particles originating in other tissues, thereby analyzing and/or isolating brain-derived cells or particles from the blood sample.

21-23. (canceled)
